(19) United States
(12) Patent Application Publication
(10) Pub. No.: US 2009/0117607 A1
(43) Pub. Date: May 7, 2009

(54) Methods For Differential Detection Of Hypoxic Tissue From A Tissue Sample Of A Mammalian Subject

(76) Inventor: Cameron J. Koch, Swarthmore, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/262,253

(22) Filed: Oct. 31, 2008

Related U.S. Application Data
(60) Provisional application No. 60/984,623, filed on Nov. 1, 2007.

(51) Int. Cl. C12Q 1/02

(52) U.S. Cl. 435/29

(57) ABSTRACT

Methods are provided for detecting levels of low oxygen in a tissue of a mammalian subject by administering non-polar halogen compounds to the mammalian subject. Detection may be accomplished using imaging methods involving fluorescence immunohistochemical imaging of the halogen compound.

### CROSS REFERENCE TO RELATED APPLICATIONS

This application claims the benefit of U.S. patent application No. 60/984,623, filed Nov. 1, 2007, which is incorporated herein by reference in its entirety.

### STATEMENT OF GOVERNMENT SUPPORT

This invention was made by government support by Grant No. CA 75285 from the National Institutes of Health. The U.S. Government has certain rights in this invention.

### FIELD

This invention generally relates to fluorine compounds and methods for detecting clinically relevant levels of low oxygen in tissue. Detection can be accomplished using imaging methods involving non-polar halogen compounds in biopsy tissue. These compounds allow the imaging of tissues and their localization to hypoxic tissues using imaging techniques such as fluorescence immunohisotchemical imaging.

### BACKGROUND

Squamous cell carcinoma of the head and neck (HNSCC) is a common and debilitating disease with 31,000 new cases and 7,500 deaths in the United States in 2006. Jemal, et al., CA-Cancer J. Clin., 2006, 56, 106-130. Treatment with curative intent involves the combination of surgery, radiation therapy, and/or chemotherapy. None-the-less, these cancers are biologically aggressive with 80% of recurrences developing within 2 years. Pazdur, Cancer Management, 7 ed., Melville, N.Y.: PRR, 2003. More aggressive combined modality treatment has resulted in five-year survival as high as 40-60%. Vokes, et al., New Engl. J. Med., 1993, 328, 184-194; Brizel, et al., New Engl. J. Med., 1998; 338, 1798-1804; Wendt, et al., J. Clin. Oncol., 1998, 16, 1318-1324; Calais, et al., J. Natl. Cancer I., 1999, 91, 2081-2086; Movsas, et al., Cancer, 2000, 89, 2018-2024. Due to the very poor prognosis for patients with recurrent disease and the morbidity of subsequent aggressive therapies, it is critical to identify patients, early in their treatment regimes who might benefit from such therapy. This would also spare patients with responsive tumors the toxicity of intensive treatment. The key to initiating this paradigm is to identify resistance factors in individual patient tumors; adjuvant therapy that modulates hypoxia, for example could then be prescribed. Studies have shown that in addition to causing radiation resistance, hypoxic tumors are more resistant to chemotherapy and are more susceptible to genetic instability. Mottram, Brit. J. Cancer, 1936, 9, 606-614; Thomlinson, et al., Br. J. Cancer, 1955, 9, 539-579; Brown, et al., Nat. Rev. Cancer., 2004, 4, 437-447; Reynolds, et al., Cancer Res., 1996, 56, 5754-5757. The latter contributes to biologically aggressive tumors with increased invasive and metastatic potential. Hypoxia is highly heterogeneous between HNSCC tumors and is not measurable using standard characteristics such as tumor grade or size; in order to individualize treatment, tumor pO₂ must be measured in individual patient tumors. Several clinical approaches have been used to accomplish this (for review see Evans, et al., Cancer Lett., 2003, 195, 1-16). Two clinically relevant “invasive” methods are needle electrodes techniques and 2-nitroimidazole binding studies, but the former can seldom be used in primary tumors of the head and neck. For the latter, the 2-nitroimidazole adduct formation in hypoxic cells can be assayed using immunohistochemistry (IHC), flow cytometry (FCM) and/or PET imaging (for example, Koh, et al., Int. J. Radiat. Oncol., Biol., Phys., 1992, 22, 199-212; Nordsmark, et al., Brit. J. Cancer, 2001, 84, 1070-1075; Ziemer, et al., Eur. J. Nucl. Med. Mol. I., 2003, 30, 259-266; Evans, et al., Brit. J. Cancer, 1995, 72, 875-882; Koch, et al., Brit. J. Cancer, 1995, 72, 869-874).

An early study of in vivo hypoxia was performed with polarographic needle electrodes in enlarged, squamous cell carcinoma-containing neck lymph nodes. Gatenby, et al., Radiol., 1983, 146, 717-719. This work demonstrated that hypoxia could be measured in a minimally invasive manner and that the resulting values were correlated with short-term treatment response. Gatenby, et al., Int. J. Radiat. Oncol., Biol., Phys., 1988, 14, 831-838. Subsequent studies using the Eppendorf pO₂ Histograph indicated that hypoxia was a predictive factor for patient outcome in HNSCC, as well as cervical cancers, soft tissue sarcomas and prostate tumors. Brizel, et al., Int. J. Radiat. Oncol., Biol., Phys., 1997, 38, 285-289; Fyles, et al., Radiother. Oncol., 1998, 48, 149-156; Brizel, et al., Cancer Res., 1996, 56, 941-943; Movsas, et al., Cancer, 2000, 89, 2018-2024. The majority of electrode measurements in head and neck sites were in enlarged lymph nodes rather than primary tumors due to the difficulty of accessing many tumors and/or making measurements safely near bone or large vessels. For patients with tumors in difficult sites and who are going to surgery, a 2-nitroimidazole hypoxia-measuring agent can be administered. These drugs bind to cells at a rate inversely proportional to the cellular pO₂. For IHC analyses, surgical biopsies made 12-48 hours after drug administration can be stained for adducts that form intracellularly.

Binding of the 2-nitroimidazole agent EF5 has been studied in several human tumor sites in order to improve understanding of in vivo hypoxia. Evans, et al., Cancer Res., 2000, 60, 2018-2024; Evans, et al., Int. J. Radiat. Oncol., Biol., Phys., 2001, 49, 587-596; Evans, et al, Clin. Cancer Res., 2004, 10, 8177-8184; Evans, et al., Cancer Res., 2004, 64, 1886-1892. Using this detection system, patterns of hypoxia can be described and the pO₂ in individual cells and/or tumor regions can be quantified. This methodology also allows examination of the spatial relationship between hypoxia and blood vessels, proliferating cells, apoptotic cells and other endpoints. Evans, et al., Am. J. Clin. Oncol., 2001, 24, 467-472. It has been previously reported that EF5 binding correlates with the level of tumor aggressiveness in supratentorial glial neoplasms and with outcome in patients with extremity soft tissue sarcoma. Evans, et al, Clin. Cancer Res., 2004, 10, 8177-8184; Evans, et al., Int. J. Radiat. Oncol., Biol., Phys., 2001, 49, 587-596. A need exists in the art for an immunohistochemistry imaging technique with improved contrast and sensitivity for imaging hypoxic tissue in cancer and other diseases, and for differential determination of hypoxic tissue and necrotic tissue in order to identify hypoxic tissue for surgical removal or for further treatment.

### SUMMARY

This invention presents methods for detecting levels of low oxygen in tissue utilizing non-polar halogen compounds in a mammalian subject. The methods of the invention provide imaging techniques involving fluorescence immunohistochemical imaging of the non-polar halogen compounds in biopsy tissues and provide the basis for sensitive and precise methods for differential detection of tissue hypoxia in the mammalian subject. The method also provides imaging of cancer or a tumor in the mammalian subject by differential detection of tissue hypoxia or tissue necrosis in the mammalian subject.

According to one aspect of the present invention, a method for detecting clinically relevant tissue hypoxia in a mammalian subject, is provided which comprises the steps of administering to the mammalian subject a compound of the formula:

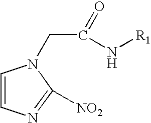

wherein R₁ is an alkyl group having up to 6 halogen atoms, wherein said alkyl group has the formula —CH₂—CX₂—CX₃, wherein X is halogen or hydrogen and at least 1 carbon atom of said alkyl group is bound with at least one halogen atom, achieving a substantially homogeneous tissue distribution of the compound in at least one tissue of interest in the mammalian subject, optimizing contrast produced by the hypoxia-dependent uptake of the compound in the tissue of the mammalian subject, removing a tissue biopsy from the mammalian subject and detecting hypoxia in the tissue biopsy by quantitative fluorescence immunohistochemical imaging of the compound in the tissue. In one aspect, the halogen atom is fluorine or bromine. The compound can comprise about five fluorine atoms. In a detailed aspect, R₁ is selected from the group consisting of —CH₂—CH₂—CH₂—F, —CH₂—CF₂—CH₂—F, —CH₂—CF₂—CHF₂, —CH₂—CHF—CH₂—F, or —CH₂—CHF—CHF₂. In a further aspect, R₁ has the formula —CH₂—CX₂—CY₃, where X is halogen or hydrogen and Y is fluorine or bromine. In a detailed aspect, R₁ is selected from the group consisting of —CH₂—CF₂—CF₃, —CH₂—CHF—CF₃ or —CH₂—CH₂—CF₃. In a further detailed aspect, R₁ is selected from the group consisting of —CH₂—CH₂—CH₂—F, —CH₂—CH₂—CF₃ or —CH₂—CF₂—CF₃. The method can further comprise distinguishing tissue hypoxia from tissue necrosis in the mammalian subject. In a further aspect, assessing hypoxia in the tissue biopsy is correlated to event-free survival and overall survival of the mammalian subject.

In one embodiment, the method further comprises identifying the hypoxic tissue for a disease state or physiological condition. In a further embodiment, the method further comprises identifying the hypoxic tissue for surgical removal or additional therapeutic treatment. The disease state or physiological condition can include, but is not limited to, diabetes, stroke, organ infarction, neonatal cerebral hypoxia, ischemic bowel disease, wound healing, granulomas, cardiac disease, organ tortion, abscesses, assessment of anti-angiogenic therapy, assessment of vascular disrupting agents, assessment of photodynamic therapy, modifications of tissue characteristics after radiation therapy, disruption of blood-brain barrier, or trauma. In one aspect, the disease state or physiological condition is cancer or solid tumor. In a detailed aspect, the disease state is head and neck squamous cell carcinoma.

### BRIEF DESCRIPTION OF THE DRAWINGS

FIGS. 1A, 1B, 1C, 1D, 1E, and 1F show EF5 Binding Patterns. Three different EF5 binding patterns were identified in HNSCC sections. (Top row—EF5 immunofluorescence; bottom row—Hoechst 33342 nuclear staining) Images A,D: Type 1 pattern: EF5 binding both centrally and in the periphery of cells, adjacent to regions of necrosis. Images B,E: Type 2 pattern: associated with extensive keratinization and characterized by a high level of EF5 binding concentrated in the periphery of cells. Images C,F: Type 3 pattern: characterized by a mixture of the two previous patterns.

FIG. 2 shows event-free survival of 22 patients with oral, oropharyngeal or laryngeal cancer. Solid line—EF5 binding <30% maximum binding, Dashed line, EF5 binding >30% maximum binding. p=0.032

FIGS. 3A and 3B show proliferation, blood vessels and EF5 binding in oral squamous cell cancer. FIGS. 3A and 3B represent the relationship between EF5 binding and Ki67 (proliferation) or CD31 (blood vessels), respectively. These images show the presence of both metabolic (chronic) and vascular mediated (acute) hypoxia. In FIG. 3A, EF5 intensity is doubled in order to more clearly delineate the patterns of hypoxia. Scale bar=2.3 mm.

### DETAILED DESCRIPTION

This invention presents methods for detecting levels of low oxygen in tissue utilizing non-polar fluorine compounds in a mammalian subject. Aspects of the present invention investigated EF5 binding in previously untreated HNSCC tumors of the oral cavity, oropharynx and larynx. Following excisional surgery, patients received varying treatments based on doctor's recommendation and patient's preferences. Despite the heterogeneity of this population, high EF5 binding was significantly associated with poor outcome. Thus, the present study has established a proof of principle for the use of this and/or non-invasive EF5-based hypoxia imaging assays to identify patients who require aggressive therapy. More extensive studies of the additional impact of patient demographics and treatment regime will be performed as well as non-invasive studies using ¹⁸F-EF5 PET imaging.

EF5, a 2-nitroimidazole hypoxia marker, was used to study the presence, levels and prognostic significance of hypoxia in primary head and neck squamous cell tumors. 22 patients with newly diagnosed squamous cell carcinoma of the oral cavity, oropharynx or larynx with at least 2 years of clinical follow-up were included in this study. Quantitative analysis of EF5 immunofluorescence was carried out and these data were compared to patient outcome. EF5 immunostaining demonstrated substantial inter-tumoral hypoxic heterogeneity. The majority of cells in all tumors were well oxygenated. Three patterns of EF5 binding in cells were identified using criteria of the cellular region that was stained (peripheral or central) and the relationship of binding to necrosis. The association between EF5 binding levels with event-free and overall survival was tested, irrespective of the pattern of cellular binding or treatment regime. Patients with tumors containing EF5 binding regions corresponding to severe hypoxia (<0.1% oxygen) had a shorter event-free survival time than patients with pO2 values >0.1% (p=0.032). Nodal status was also predictive for outcome. These data illustrate the potential utility of EF5 binding based on quantitative immunohistochemistry of tissue pO2 and provide support for development of non-invasive hypoxia PET studies with ¹⁸F-EF5. See, for example, U.S. Pat. Nos. 5,540,908, 5,843,404, and 6,252,087, each incorporated herein by reference in their entireties.

This invention relates to methods for detecting levels of low oxygen in tissue using non-polar halogen compounds, for example, fluorine compounds. Detection may be accomplished using imaging methods involving fluorescence immunohistochemical imaging of non-polar compounds for example, fluorine compounds such as EF1,3, EF3, or EF5 compounds. These compounds allow the imaging of tissues and their localization using fluorescent imaging techniques. For example, a group of compounds have been prepared which when activated by reductive metabolism, bind to hypoxic cells. This reductive metabolism and binding increase as the oxygen concentration of cells decreases, which enables these compounds to be used as indicators of hypoxia. The methods are useful on tissue biopsies of cancer or solid tumors, for example head and neck squamous cell carcinoma.

Patterns and Levels of Hypoxia in Head and Neck Squamous Cell Carcinomas and their Relationship to Patient Outcome

22 patients with newly diagnosed HNSCC were enrolled on the EF5 trial and were included in this study (Table 2). All patients had a minimum of 2 years follow-up or experienced recurrence, metastasis or death before 2 years. There were 4 female and 18 male patients with a mean age of 61.6 years (range 45-77 years). Seventeen patients had oral cavity or oropharyngeal tumors and 5 patients had laryngeal cancers. Tumor morphologies ranged from well- to poorly-differentiated; 16 patients had stage 4, 3 patients had stage 3, 2 patients had stage 2 and 1 patient had stage 1 disease (AJCC Cancer Staging, 6^{th} edition). Two patients received chemotherapy and 14 patients received radiation therapy following surgical excision. One patient each experienced EF5-related grade 1 and 2 toxicity. There were no episodes of EF5-related grade 3 or 4 toxicity during the 30 days following EF5 administration.

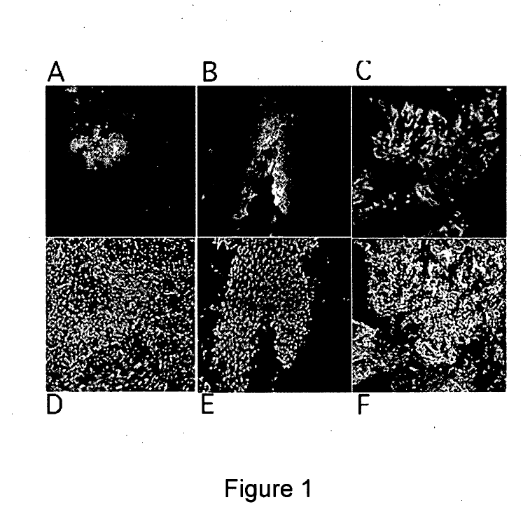

Three EF5 binding patterns, designated as Types 1-3 were seen (FIG. 1a-c). These patterns were subjectively differentiated from each other based on the cellular location of binding, as described above. These binding patterns varied within and between patients and there was no apparent relationship between the most common binding pattern in each tissue section and the level of tumor differentiation. Binding patterns did not correlate with any outcome measure in this small sample.

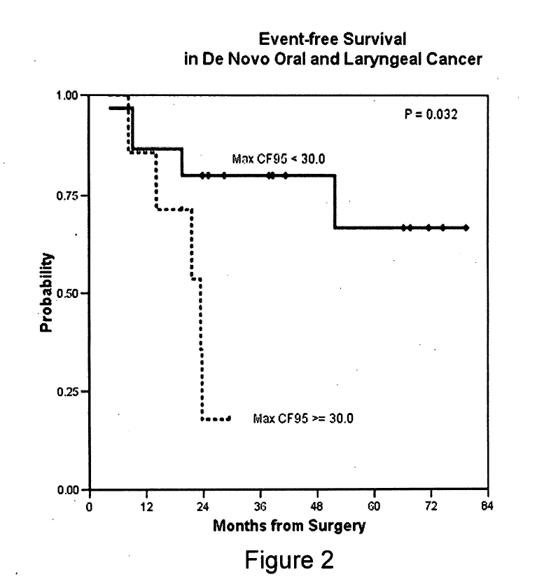

The prognostic importance of tumor stage, lymph node status, and EF5 binding level (average or maximum CF₉₅) was explored by testing the association of these variables with event-free (EFS) and overall survival (OS). At a median follow-up of 38.7 months, there were 9 events and 6 deaths. These 9 events included: 3 patients with metastatic disease, 5 patients with local disease recurrence and 1 patient with both. The 3-year EFS rate (±SE) was 62.6% (±10.5%) for the entire study group. Higher nodal stage (p=0.039) and tumors with poorly differentiated regions (p=0.055) were associated with shorter EFS. Severe hypoxia was not associated with higher nodal stage (N0-N1 26.7% vs. N2-N3 42.9%, p=0.63 Fisher's exact test). Of the 5 subcategories listed in Table 1 to describe hypoxia, only severe hypoxia (max CF₉₅>30% maximum binding; p=0.032) was associated with shorter EFS (FIG. 2). Patients with CF95≧30% maximum binding had shorter EFS as compared to those with CF₉₅<30; this difference did not reach statistical significance after adjusting for nodal stage (p=0.08).

TABLE 1
Descriptor and average relationship
between EF5 binding and tissue pO2

| | Maximum EF5 | pO2 | pO2 |
|---|---|---|---|
| Descriptor | binding (%) | (%) | (mm Hg) |
| Normoxia | 1 | 10 | 76 |
| Modest (mild) hypoxia | 3 | 2.5 | 19 |
| Moderate hypoxia | 10 | 0.5 | 3.8 |
| Severe hypoxia | 30 | 0.1 | 0.76 |
| Anoxia | 100 | 0 | 0 |

Methods for detecting clinically relevant tissue hypoxia are illustrated in Table 1. Normoxia is indicated at a maximum EF5 binding of 1%. Modest hypoxia is indicated at a maximum EF5 binding of 1%. Moderate hypoxia is indicated at a maximum EF5 binding of 10%. Severe hypoxia is indicated at a maximum EF5 binding of 30%. Anoxia is indicated at a maximum EF5 binding of 100%.

The 3-year OS rate (±SE) was 67.4% (±11.4%) for the entire study group. Although OS rates were lower for patients with more severe hypoxia, these did not reach statistical significance. This may be due to the variable treatments initially and following recurrence, small study size and number of treatment failures (Table 2). Nodal stage (N0-N1 vs. N2-N3) was the only factor associated with overall survival (univariate analysis; p=0.024).

TABLE 2
Demographic, pathologic, and EF5 binding data

| | | | | | | | EF5 | EF5 | Hypoxia |
|---|---|---|---|---|---|---|---|---|---|
| | | | T | N | | Initial | binding | binding | grouping |
| Patient | | Extent of | (tumor) | (node) | Initial | radiation | average | maximum | (Based on |
| no. | Site | differentiation | stage* | stage* | chemotherapy | therapy | CF₉₅ | CF95 | CF₉₅ maximum |
| 24 | Tongue | M | 4a | 1 | | X | 0.4 | 1.2 | NOR |
| 17 | Tongue | M | 4b | 3 | X | X | 1.3 | 1.3 | NOR |
| 15 | Floor of | W | 4a | 0 | | | 0.8 | 1.4 | NOR |
| | mouth | | | | | | | | |
| 14 | Floor of | M | 3 | 0 | | | 0.9 | 1.4 | NOR |
| | mouth | | | | | | | | |
| 54 | Maxilla | P | 2 | 0 | X | X | 1.9 | 2.8 | NOR |
| 7 | Retromolar | M | 4a | 1 | | X | 1.1 | 6.0 | MLD |
| | trigone | | | | | | | | |
| 21 | Alveolus | M | 4a | 0 | | | 4.2 | 7.9 | MLD |
| 47 | Mandible | W | 4a | 0 | | X | 5.6 | 8.2 | MLD |
| 26 | Retromolar | M | 4a | 0 | | X | 6.2 | 8.9 | MLD |
| | trigone | | | | | | | | |
| 85 | Tongue | WM | 4a | 2 | | X | 6.1 | 10.0 | MLD |
| 67 | Supraglottic | P | 4a | 2b | | | 6.4 | 16.4 | MOD |
| | larynx | | | | | | | | |
| 4 | Floor of | MP | 4a | 0 | | | 10.8 | 17.8 | MOD |
| | mouth | | | | | | | | |
| 80 | Supraglottic | MP | 3 | 1 | | | 16.0 | 19.4 | MOD |
| | larynx | | | | | | | | |
| 81 | Supraglottic | M | 4a | 2 | | X | 19.6 | 23.1 | MOD |
| | larynx | | | | | | | | |
| 61 | Tongue | M | 2 | 0 | | X | 18.7 | 25.4 | MOD |
| 63 | Maxilla | M | 4a | 0 | | X | 12.5 | 30.7 | SEV |
| 68 | Buccal | W | 1 | 0 | | | 26.0 | 35.8 | SEV |
| | mucosa | | | | | | | | |
| 83 | Glottic | M | 4a | 0 | | X | 14.2 | 36.0 | SEV |
| | larynx | | | | | | | | |
| 48 | Tongue | M-P | 4a | 2 | | X | 23.0 | 61.7 | SEV |
| 5 | Tongue | M | 4 | 2b | | X | 36.9 | 66.0 | SEV |
| 88 | Buccal | P | 4a | 2b | | X | 38.5 | 66.5 | SEV |
| | mucosa | | | | | | | | |
| 75 | Supraglottic | P | 3 | 0 | | | 34.5 | 66.6 | SEV |
| | larynx | | | | | | | | |

Abbreviations: CF95 = 95% of EF5 values in the image were at or less than cube reference binding value; Diff = tumor differentiation; M = moderately differentiated, P = poorly differentiated, W = well differentiated; NOR = normoxia; MLD = mild/moderate hypoxia; MOD = moderate hypoxia; SEV = severe hypoxia.

TABLE 3
Distribution of maximum CF95 values by patient characteristics

| | | | | | | | | Fisher’s |
|---|---|---|---|---|---|---|---|---|
| | No. of | Maximum CF₉₅ | | Maximum CF₉₅ < 30 | | Maximum CF₉₅ >= 30 | | exact |
| | patients | Mean SD | | No. % | | No. % | | test p |
| All patients | 22 | 23.38 | 22.90 | 15 | 68.2 | 7 | 31.8 | |

Gender

| | | | | | | | | |
|---|---|---|---|---|---|---|---|---|
| Men | 18 | 26.74 | 23.95 | 11 | 61.1 | 7 | 38.9 | |
| Women | 4 | 8.27 | 6.89 | 4 | 100.0 | 0 | 0.0 | 0.26 |

Age (y)

| | | | | | | | | |
|---|---|---|---|---|---|---|---|---|
| <60 | 11 | 25.66 | 23.06 | 7 | 63.6 | 4 | 36.4 | |
| ≧60 | 11 | 21.11 | 23.62 | 8 | 72.7 | 3 | 27.3 | 1.00 |

Differentiation

| | | | | | | | | |
|---|---|---|---|---|---|---|---|---|
| Moderate | 10 | 20.00 | 20.79 | 7 | 70.0 | 3 | 30.0 | |
| Well, well to moderate | 5 | 12.65 | 13.33 | 4 | 80.0 | 1 | 20.0 | |
| Moderate to poor, poor | 7 | 35.87 | 27.77 | 4 | 57.1 | 3 | 42.9 | 0.72 |

Nodal stage

| | | | | | | | | |
|---|---|---|---|---|---|---|---|---|
| N0-N1 | 15 | 17.96 | 18.33 | 11 | 73.3 | 4 | 26.7 | |
| N2-N3 | 7 | 35.00 | 28.63 | 4 | 57.1 | 3 | 42.9 | 0.63 |

Abbreviation: CF₉₅ = 95% of EF5 values in the image were at or less than cube reference binding value.

Utility of EF5 Binding Based on Quantitative Immunohistochemistry of Tissue pO2

There is substantial support for the observation that hypoxia leads to treatment resistance and is a prognostic factor for outcome in patients with HNSCC. Gatenby, et al., Int. J. Radiat. Oncol., Biol., Phys., 1988, 14, 831-838; Brizel, et al., Int. J. Radiat. Oncol., Biol., Phys., 1997, 38, 285-289; Kaanders, et al., Int. J. Radiat. Oncol., Biol., Phys., 2002, 52, 769-778. The earliest observations suggesting that hypoxia could be measured in this disease were based on the use of polarographic needle electrodes. Gatenby, et al., Radiol., 1983, 146, 717-719. Almost all of the HNSCC patients studied with this technique had measurements made in enlarged draining lymph nodes. Electrode measurements of pO₂ in primary tumors and/or lymph node metastases found that hypoxia was correlated with EFS and OS. Brizel, et al., Int. J. Radiat. Oncol., Biol., Phys., 1997, 38, 285-289. It is often necessary to perform needle electrode measurements for HNSCC in the lymph node rather than in the primary tumor because (1) laryngeal and pharyngeal tumors are usually inaccessible to needle electrodes and (2) oral cavity tumors are often closely associated with bone. In the few patients where primary tumors and lymph node metastases were measured concurrently, a concordance between the pO₂ levels in each site was reported; Becker et al demonstrated a correlation between the median pO₂s of primary tumors and lymph node metastases in a small group of patients (n=30). Becker, et al., Int. J. Radiat. Oncol., Biol., Phys., 1998, 42, 35-41. Lymph node pO₂ measurements have not been made in patients with normal sized lymph nodes despite the observation that normal sized nodes can contain regions of necrosis based on imaging (Dr. L. Loevner, University of Pennsylvania in a personal communication, 2006). The use of EF5 in patients going to surgery (biopsy or therapeutic resection) circumvents these limitations. Since many patients undergoing therapeutic surgical resection will also undergo an ipsilateral (and sometimes bilateral) nodal dissection, it is possible to assay both enlarged and normal sized nodes.

In addition to papers describing hypoxia in HNSCC, there has been great interest in measuring hypoxia in tumors using endogenous hypoxia markers such as CAIX, Glut-1 and HIF. There are advantages and limitations to this approach; several studies have shown a limited co-localization of endogenous markers with pimonidazole, another 2-nitroimiazole hypoxia detection agent but other studies with this agent have found none (for example, see Hoogsteen, et al., Radiother. Oncol., 2005, 76, 213-218). Arcasoy, et al., Lab. Invest., 2002, 82, 911-918; Koukourakis, et al., Clin. Cancer Res., 2001, 7, 3399-3403; Vordermark, et al., Strahlenther Onkol., 2003, 179, 801-811. One study using EF5 supports co-localization with VEGF protein (in situ hybridization). Ziemer, et al., Neoplasia, 2001, 3, 500-508.

There have been only a few IHC-based studies that prospectively evaluate the association of hypoxia and outcome in cancer patients. In a large study of 127 patients with cervix cancer, pimonidazole binding was not predictive of patient outcome. Nordsmark, et al, Radiother. Oncol., 2006, 80, 123-131. In a HNSCC study by Kaanders, HNSCC patients with larger regions of pimonidazole binding had a better 2-year local control rate after treatment with ARCON (Accelerated Radiation Therapy with Oxygen and Nicotinamide) than patients with hypoxic tumors treated with standard therapy. Kaanders, et al., Int. J. Radiat. Oncol., Biol., Phys., 2002, 52, 769-778. This study did not test a therapeutic and differs from this work in several other ways; the tumor characteristics were less stringent in the present study; patients with Stage I and II disease were included, tissue analyses were performed on tumor resection vs. biopsy, there was a longer time between drug infusion and biopsy (pimonidazole 2 hours, EF5 approximately 24 hours), and there were substantial differences in image analysis. In the Kaander's paper regions of pimonidazole binding were first dichotomized into presence vs. absence and then the fractional tissue area with binding was reported. In the present study, EF5 binding was analyzed as a continuous variable with the cut-off value for outcome based on tissue pO₂, e.g patients with severe hypoxia (CF₉₅>30%, pO2<0.1%) had a shorter EFS time than patients without severely hypoxic tumor regions. The present study also reports that nodal status was important to patient outcome whereas this relationship was not significant in the pimonidazole study. These findings of an association between nodal involvement and poor patient outcome supports the reports in the literature that nodal status is the most important predictor of outcome in HNSCC. Leemans, et al., Cancer, 1994, 73, 187-190; Ljumanovic, et al., Eur. J. Radiol., 2006, 60, 58-66.

This study was specifically designed to describe the presence, levels and patterns of EF5 binding in HNSCC and to explore the possibility that the level of binding was a prognostic factor for outcome. Due to the study design and the small number of patients studied, it was not possible to determine whether EF5 binding was a predictive factor for outcome, e.g. that targeting hypoxia would modify patient outcome. It should be noted however, that although the number of patients presented herein is small (n=22), it is similar to studies using the Eppendorf needle electrode.

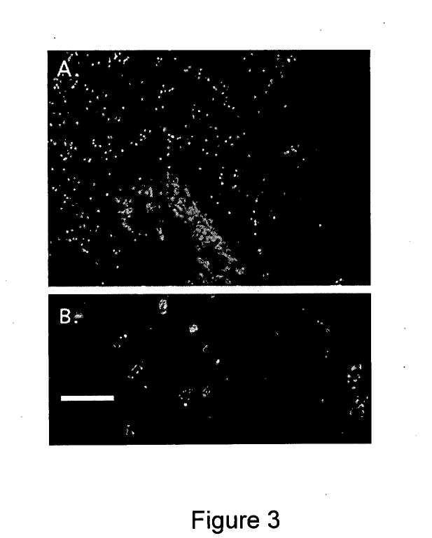

Intriguing observations from this study include the varied patterns of EF5 binding in HNSCC. Type 1 binding pattern is most similar to EF5 binding in sarcomas and brain tumors and Type 2 binding is most reminiscent of the EF5 binding patterns described in normal human skin. Evans, et al., Int. J. Radiat. Oncol., Biol., Phys., 2001, 49, 587-596; Evans, et al, Clin. Cancer Res., 2004, 10, 8177-8184; Evans, J. Invest. Derm., 2006. Using pimonidazole, similar patterns of binding have been reported. Wijffels, et al., Brit. J. Cancer, 2000, 83, 674-683; Azuma, et al., Clin. Cancer Res., 2003, 9, 4944-4952. These binding patterns for pimonidazole have been described in cervix cancer, but the classified patterns are on a larger scale (mm) than discussed herein (μm). Azuma et al have investigated the significance of pimonidazole binding in what is referred to as EF5 “Type 2” binding. They have performed studies to better understand the relationship between pimonidazole binding and cellular differentiation (involucrin expression) and they report that pimonidazole and involucrin do not co-localize in cervix cancer. Azuma, et al., Clin. Cancer Res., 2003, 9, 4944-4952. Based on these observations, they conclude that involucrin is a hypoxia-inducible protein, but its expression is altered by cellular differentiation. The present studies performed in human skin, which contain regions of squamous cell differentiation, suggests that “Type 2” EF5 binding is dominantly a reflection of tissue pO₂ rather than expression of other cellular proteins, state of differentiation or an artifact of elevated reductase enzymes. Evans, J. Invest. Derm., 2006. This conclusion is supported by images of the spatial relationship between EF5 binding and CD31 staining (blood vessels) or Ki67 staining (proliferation) (FIG. 3). In skin, as well as in tumors, there is an inverse location between EF5 binding and proliferation or blood vessels. The significance (as well as mechanism for development) of these binding patterns remains unknown. The continued controversy over the relationship between hypoxia, differentiation, and tumor biology warrants additional study. The current subjectivity of this evaluation and the small number of patients studied leave open the significance of patterns of EF5 binding in HNSCC. Imaging studies using newer analytic methods will help us determine whether EF5 in the entire cell and/or the cell cytoplasm is clinically relevant for patient outcome in HNSCC. Quantitative analyses with pattern recognition of these images is being developed so that these questions can be addressed more fully.

Aspects of the present invention have demonstrated the potential clinical utility of EF5 staining analysis as a prognostic factor in patients with HNSCC, e.g., high EF5 binding is associated with shorter EFS. The association between hypoxia and poor outcome has been recognized in vitro for over 30 years and in humans for 20. Yet, there are only a handful of small trials demonstrating that targeting hypoxia is effective in modifying tumor response. Indeed, many of such trials have been negative. For the purpose of optimizing the likelihood of success of these agents in the future, the studies of misonidazole as a hypoxic radiation sensitizer in the 1970s and 1980s are most instructive. Almost all of these trial were negative and some possible reasons for these failures have been described. Coleman, et al., Int. J. Radiat. Oncol., Biol., Phys., 1990, 18, 389-393; Koch, et al., Biochem. Pharmacol., 1993, 46, 1029-1036. Nonetheless, when the trials were analyzed using meta-analysis methodology (essentially increasing the study population), the effectiveness of misonidazole as a hypoxic cell sensitizer in HNSCC could be identified in a subset of patients. Overgaard, Radiother. Oncol., 1992, 24, S64. These results indicate that small clinical trials will not be successful unless patients with hypoxic tumors can be identified. It is clear that the best way to proceed in developing hypoxia as a predictive factor is to identify and treat patients with this tumor microenvironmental characteristic. These types of studies have not yet been performed on a large scale, likely due to the cost and/or logistics of evaluating tumor hypoxia in each patient before treatment is prescribed.

Based on the observations reported herein, studies are being pursued using PET evaluation of HNSCC using ¹⁸F-EF5 binding. The unique characteristic of EF5 and ¹⁸F-EF5 pair is that the drug used in both is identical in chemical structure. Thus, IHC-based analyses can be used to validate ¹⁸F EF5 images. The confirmation that IHC hypoxia measurements using EF5 are prognostically important in human HNSCC vastly improves the likelihood that ¹⁸F-EF5 imaging will succeed in this regard. The use of ¹⁸F EF5 imaging for prognostic purposes becomes particularly relevant in the context of the evaluation of treatment planning in patients for whom surgery is not clinically indicated. Analysis of tumor hypoxia should allow a more detailed examination of patient subgroups in investigational treatment trials. A recent study reported by Rischin et al illustrated this concept in 45 patients with HNSCC studied with ¹⁸F-fluoromisonidazole PET. Rischin, et al., J. Clin. Oncol., 2006; 24, 2098-2104. Following imaging, patients were treated with adjuvant cisplatin either with infusional fluorouracil or the hypoxia cytotoxin, tirapazamine. Patients with hypoxic tumors treated with tirapazamine did as well as those with oxic tumors, irrespective of treatment. These three groups of patients had a statistically improved outcome (local-regional failure) than patients with hypoxic tumors treated in the standard manner. Another advantage of non-invasive imaging is that it potentially allows repeated evaluation of tumor hypoxia over its entire tumor volume and over the time course of therapy.

The results reported herein support the use of EF5 as a measure of hypoxia in HNSCC. Outcome data show an association between high EF5 binding and low EFS, specifically in tumors-containing regions of severe hypoxia. Larger studies utilizing unlabelled EF5, as reported herein and/or PET imaging of ¹⁸F-EF5 are being performed or planned at several institutions worldwide.

Materials and Methods

Human Subjects. The Institutional Review Board of the University of Pennsylvania Clinical Trials and Scientific Monitoring Committee at the University of Pennsylvania Abramson Cancer Center and the Cancer Therapeutics Evaluation Program of the National Cancer Institute approved this prospective trial of EF5. All patients signed an approved consent form for both the study and for privacy (HIPPA). Patients of all ethnic and gender groups were included. Eligible patients were those undergoing resection or biopsy of a HNSCC as clinically indicated. Exclusion criteria included patients with a history of grade III or IV peripheral neuropathy, pregnant or nursing patients and patients <18 years old. Coleman, et al., Int. J. Radiat. Oncol., Biol., Phys., 1990, 18, 389-393.

EF5 Administration. The National Cancer Institute, Division of Cancer Treatment supplied EF5 in 100 mL vials containing an aqueous solution of 3 mg/mL EF5 also containing 5% dextrose and 2.4% ethanol. This EF5 solution was administered through a peripheral IV catheter, 24-48 hours before surgery at a rate no greater than 350 mL/hr and to a total dose of 9-21 mg/kg. Blood samples for drug levels were obtained before and 1 hour after the completion of drug administration, and at the time of surgery. Calculation of drug time/dose concentration (area under the curve, AUC) allowed the absolute EF5 tissue binding level to be corrected for variations in drug dose and exposure time between patients. Koch, et al., Cancer Chemother. Pharmacol., 2001, 48, 177-187.

Tissue Acquisition and processing for EF5 Binding. 24-48 hours after completion of drug administration, tumors were resected or biopsied and placed in sterile, iced EXCELL 610 medium (JRH Biosciences, Lenexa, Kans.). In collaboration with the pathologist, tissue samples deemed unnecessary for clinical diagnosis were taken to the lab for processing and analysis.

IHC staining methods and quantitative fluorescence microscopy of EF5 in in situ tumor sections and EF3 in tissue cubes for cube reference binding (CRB) were performed as reported previously. Evans, et al., Cancer Res., 2004, 64, 1886-1892. CRB determination is an in vitro technique where tissue cubes are incubated in 200 μM EF3, a sister drug of EF5, under controlled hypoxic conditions for 3 hours. The goal of this procedure was to determine the maximum level of EF3 binding possible for each patient's tumor. This technique allowed normalization of the in situ EF5 binding to each tumor's maximum binding value after correction for the AUC. Koch, et al., Cancer Chemother. Pharmacol., 2001, 48, 177-187. These values were used to calculate the maximum and average ‘% CRB’ value for each tumor, as described previously and briefly below. Evans, et al., Cancer Res., 2000, 60, 2018-2024. Thus, EF5 binding values for each tumor tissue are reported on a scale of 0-100% allowing for comparisons within and between tumors.

Quantitative Image analysis. The quantitative analysis of tumor sections stained for EF3/EF5 has been previously described. Evans, J. Invest. Derm., 2006; Evans, et al., Int. J. Radiat. Oncol., Biol., Phys., 2001, 49, 587-596; Busch, et al., Adv Exp Med. Biol., 2003, 510, 37-43. Briefly, tissue sections were counterstained with Hoechst 33342 to identify the location of intact nuclei. These images were used to create bitmaps in order to generate binary masks of viable tissue regions. Masked regions of each section were analyzed for EF5-dependent immunofluorescence intensity using routines written in MatLab (The MathWorks, Inc., Natick, Mass.). EF5 intensity values of these regions were corrected for tissue section thickness, camera exposure time, relative lamp intensity, EF5 drug exposure (AUC) and CRB. Evans, et al., Cancer Res., 2000, 60, 2018-2024; Evans, et al., Int. J. Radiat. Oncol., Biol., Phys., 2001, 49, 587-596; Evans, et al, Clin. Cancer Res., 2004, 10, 8177-8184; Koch, et al., Cancer Chemother. Pharmacol., 2001, 48, 177-187. Data were then summarized by generating histograms of all positive pixels in the masked image, which were then converted into a cumulative frequency plot. The number of pixels at or below a given binding level can be identified from this plot. For example, CF₉₅=20% would mean that 95% of the EF5 values in the image were at or below 20% of CRB. Terms to describe tissue oxygenation based on EF5 studies have been approximated by data from in vitro studies (Table 1). Koch, Method Enzymol., 2002, 352, 3-31. Normoxic conditions correspond to a CF₉₅≈1%; for modestly/mildly hypoxic conditions, CF₉₅≈3%; for moderately hypoxic conditions, CF₉₅≈10%; for severely hypoxic conditions, CF₉₅≈30%, and for anoxia, the CF₉₅100%. The pO₂ corresponding to these descriptive terms is found in Table 1. Since all patients had at least two pathologically confirmed tumor tissue samples stained for EF5, both the average (average CF₉₅) and the brightest binding (maximum CF₉₅) of all tissue sections studied for each patient are reported.

All tissue sections that were analyzed for EF5 binding were reviewed by a pathologist (PZ) at the Hospital of the University of Pennsylvania in order to confirm that the tissue section contained tumor. Tissue sections that contained <20% viable tumor or >80% normal tissue or necrosis were eliminated from further analysis. The histologic diagnosis of the tumor in the EF5-stained tissue sections was compared to the pathologic diagnosis based upon paraffin-embedded tissue sections. None of the EF5-stained tissue sections had a diagnosis that differed from that rendered clinically.

EF5 Binding Patterns. Images of each tissue specimen containing mild, moderate or severe hypoxia (EF5 binding >3% of CRB) were independently and subjectively examined by two authors (SME, KD) and then assigned a binding pattern. Disagreement between the two evaluators led to further discussion and consensus agreement. Binding in both the peripheral and central portion of each cell was referred to as Type 1 and slides that contained only this pattern were classified as Type 1. Type 1 binding was often seen adjacent to regions of necrosis. Binding that was dominantly peripheral in the cell was called Type 2 and slides that contained only this binding pattern were referred to as Type 2. If a tissue section contained regions of both patterns, the slide was considered to be Type 3 (FIG. 1).

Tumor staging and patient outcome. The final tumor stage was based on pathological examination of the surgical specimen, and patient outcome was confirmed by the treating physician (AAC, GSW, SMH, HQ), based on routine clinical follow-up.

Statistical Methods. Descriptive statistics (frequency, percentage, mean and standard deviation) were used to report distributions of patient, tumor and EF5 binding variables. Association between categorical variables was evaluated by Fisher's exact test. Event-free survival (EFS) was defined from date of EF5 administration to date of a documented event (recurrence, metastasis or death due to any cause) or last patient contact. Overall survival (OS) was defined from date of surgery to date of death due to any cause or last patient contact. EFS and OS were estimated by the method of Kaplan and Meier. Kaplan, et al., J. Am. Stat. Assoc., 1958, 457-481. The magnitude of the effect of patient, tumor and EF5 binding variables on EFS and OS was assessed by the hazard ratio, 95% confidence interval and Wald statistic based on the Cox proportional hazards model. Cox, et al., Analysis of Survival Data, New York: Chapman and Hall, 1990. In the case of tumor differentiation and binding patterns, estimation and significance testing based on the Cox model was not possible due to the small number of patients and events in categories of this variable (e.g., there were no events in 5 patients with well or moderate-to-well differentiation). Thus statistical significance was determined by the log-rank test. Statistical significance was defined as P<0.05.

All publications and patent applications cited in this specification are herein incorporated by reference in their entirety for all purposes as if each individual publication or patent application were specifically and individually indicated to be incorporated by reference for all purposes.

Those skilled in the art will appreciate that numerous changes and modifications may be made to the preferred embodiments of the present invention, and that such changes and modifications may be made without departing from the spirit of the invention. It is, therefore, intended that the spirit and scope of the appended claims should not be limited to the description of the preferred embodiments contained herein, but, that the appended claims cover all such equivalent variations as fall within the true spirit and scope of the invention.

What is claimed:

1. A method for detecting clinically relevant tissue hypoxia in a mammalian subject, comprising the steps of:
administering to the mammalian subject a compound of the formula: 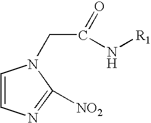
wherein R₁ is an alkyl group having up to 6 halogen atoms, wherein said alkyl group has the formula —CH₂—CX₂—CX₃, wherein X is halogen or hydrogen and at least 1 carbon atom of said alkyl group is bound with at least one halogen atom,
achieving a substantially homogeneous tissue distribution of the compound in at least one tissue of interest in the mammalian subject,
optimizing contrast produced by the hypoxia-dependent uptake of the compound in the tissue of the mammalian subject,
removing a tissue biopsy from the mammalian subject and
detecting hypoxia in the tissue biopsy by quantitative fluorescence immunohistochemical imaging of the compound in the tissue.

2. The method of claim 1 further comprising distinguishing tissue hypoxia from tissue necrosis in the mammalian subject.

3. The compound of claim 1 wherein the halogen atom is fluorine or bromine.

4. The compound of claim 1 wherein R₁ is selected from the group consisting of —CH₂—CH₂—CH₂—F, —CH₂—CF₂—CH₂—F, —CH₂—CF₂—CHF₂, —CH₂—CHF—CH₂—F, or —CH₂—CHF—CHF₂.

5. The method of claim 1 comprising about five fluorine atoms.

6. The method of claim 1 wherein R₁ has the formula —CH₂—CX₂—CY₃, where X is halogen or hydrogen and Y is fluorine or bromine.

7. The method of claim 6 wherein R₁ is selected from the group consisting of —CH₂—CF₂—CF₃, —CH₂—CHF—CF₃ or —CH₂—CH₂—CF₃.

8. The method of claim 1 wherein R₁ is selected from the group consisting of —CH₂—CH₂—CH₂—F, —CH₂—CH₂—CF₃ or —CH₂—CF₂—CF₃.

9. The method of claim 1, further comprising identifying the hypoxic tissue for a disease state or physiological condition.

10. The method of claim 9, further comprising identifying the hypoxic tissue for surgical removal or additional therapeutic treatment.

11. The method of claim 9 wherein the disease state or physiological condition is diabetes, stroke, organ infarction, neonatal cerebral hypoxia, ischemic bowel disease, wound healing, granulomas, cardiac disease, organ tortion, abscesses, assessment of anti-angiogenic therapy, assessment of vascular disrupting agents, assessment of photodynamic therapy, modifications of tissue characteristics after radiation therapy, disruption of blood-brain barrier, or trauma.

12. The method of claim 9 wherein the disease state or physiological condition is cancer or solid tumor.

13. The method of claim 12 wherein the disease state is head and neck squamous cell carcinoma.

14. The method of claim 12, wherein assessing hypoxia in the tissue biopsy is correlated to event-free survival and overall survival of the mammalian subject.
